(19) United States
(12) Patent Application Publication
(10) Pub. No.: US 2024/0112500 A1
(43) Pub. Date: Apr. 4, 2024

(54) APPARATUS AND METHOD FOR ESTIMATING DISABILITY INFORMATION BASED ON USER IMAGE DATA ANALYSIS

(71) Applicant: DOT INCORPORATION, Seoul (KR)

(72) Inventors: Ji Ho KIM, Incheon (KR); Ju Yoon Kim, Gwangmyeong-si (KR); Hyeon Cheol Park, Gwangmyeong-si (KR)

(21) Appl. No.: 17/994,311

(22) Filed: Nov. 26, 2022

(30) Foreign Application Priority Data
Sep. 29, 2022 (KR) 10-2022-0124073

(51) Int. Cl. G06V 40/20; G06V 20/52; G06V 40/18

(52) U.S. Cl. CPC G06V 40/25; G06V 20/52; G06V 40/18; G06V 2201/07

(57) ABSTRACT

Provided are a disability information estimation apparatus and method, and the disability information estimation method based on disability information includes obtaining first image data of a user, extracting user characteristic information including at least one of appearance characteristics, behavior characteristics, and object characteristics associated with the user from the first image data, and inferring disability information including a disability type and a disability degree of the user on the basis of the user characteristic information.

### CROSS REFERENCE TO RELATED APPLICATION

This application claims priority from and the benefit of Korean Patent Application No. 10-2022-0124073, filed on Sep. 29, 2022, which is hereby incorporated by reference for all purposes as if fully set forth herein.

### BACKGROUND

### Field

The disclosure relates to an apparatus and method for evaluating disability information based on user image data analysis.

### Discussion of the Background

Due to the recent 4^{th} industrial revolution and the development of IT technology, unmanned devices (e.g., kiosks or the like), which replace conventional workers in providing various information and services, have appeared in large numbers, and the frequency of their use is increasing worldwide. However, since most of the current unmanned devices mainly are produced, disseminated, and operated towards a target of non-disabled users and users of younger age groups, it is hardly difficult for the information vulnerable users, such as disabled users and elderly users, to access and use the services provided by the current unmanned devices.

In this regard, the number of disabled people accounts for about 15% of the world's population, reaching 1 billion people worldwide. In addition, the aging population has been intensified from countries of G20, and thus, it is expected that the aged population ratio reaches about 17% of the world's population by the year of 2050. After all, in the future, the disabled people or the elderly may increase to about 3 out of 10 of the total population. In addition, considering that the adoption of unmanned devices is an inevitable trend that no one can resist due to the lack of service workers, it is expected that information inequality in information vulnerable users, such as the disabled users and the elderly users, continues to intensify and becomes a big social issue.

Meanwhile, in Korea, reflecting those trends, revised bills of the Framework Act on National Informatization and the Discrimination Disability Act have been recently approved. The main contents of the revised bills are that disabled users are allowed to access and use services on an equal basis with non-disabled users. Thus, it is evaluated that the discrimination against the disabled users be abolished in the use of unmanned terminals.

However, as the automation level of unmanned devices gradually increases and the range of services that can be replaced by the unmanned devices expands, the unmanned devices tend to be actively introduced in stores and public places. In contrast to the above tendency, it is still hardly difficult for the information vulnerable users to access to the unmanned devices, and the unmanned devices still continue to be a barrier to the aforementioned information vulnerable users as the unmanned devices are operated on the premise that the users are well-acquainted with the usage of the unmanned device.

Therefore, there is a need to develop an unmanned device in which elements related to a user interface and user experience can be provided in a responsive or customized manner in consideration of the usage environment of the unmanned device and the user characteristics, so that most users, including the information vulnerable users, can use the unmanned device without difficulty. In addition, there is a need to develop a technique that can analyze disability information of the user who accesses or uses the unmanned device.

The background art of the disclosure is disclosed in Korean Patent Registration No. 10-1480326.

### SUMMARY

The disclosure is directed to providing an apparatus and method for evaluating disability information based on user image data analysis, capable of estimating the existence of disability, a disability type, a disability degree, or the like of a user in view of characteristics such as an appearance, a behavior, an object, or the like reflected in image data of the corresponding user.

The disclosure is also directed to providing a customized information providing system capable of detecting a situation in which a user has difficulty in manipulation of an information terminal such as a kiosk, an unmanned device, or the like, on the basis of an analysis result of image data for a user interacting with the corresponding information terminal, and assisting the user in response thereto.

However, the technical problems to be achieved by embodiments of the disclosure are not limited to the technical problems as described above, and other technical problems may exist.

According to an aspect of an embodiment, there is provided a disability information estimation method based on user image data analysis, the method including obtaining first image data of a user, extracting user characteristic information including at least one of appearance characteristics, behavior characteristics, or object characteristics associated with the user from the first image data, and inferring disability information including at least one of a disability type or a disability degree of the user on the basis of the user characteristic information.

In an embodiment, the disability type may include at least one of a visual impairment type, a hearing-impaired type, or a physical disability type.

In an embodiment, the disability information estimation method based on user image data analysis may include setting at least one of a first element associated with a user input application method of an information terminal to be manipulated by the user or a second element associated with an information output method of the information terminal on the basis of the disability information.

In an embodiment, in the extracting of the user characteristic information, whether the user accesses the information terminal may be determined, and the user characteristic information may be derived according to the determination result.

In an embodiment, the disability information estimation method based on user image data analysis may include obtaining second image data obtained by capturing an image of the user who manipulates the information terminal, extracting the behavior characteristics from the second image data, and evaluating a user's manipulation ability for the information terminal on the basis of the extracted behavior characteristics.

In an embodiment, in the obtaining of the first image data, at least one of image data captured through a camera module provided in the information terminal or image data obtained by an infrastructure configured to capture an image of a target space, in which the user is located, may be received as the first image data.

In an embodiment, the appearance characteristics include at least one of height information or posture information of the user.

In an embodiment, the behavior characteristics may include information on at least one of a user's gait, gaze, or movement of a preset body part.

In an embodiment, the object characteristics may include information on clothes worn by the user, items carried by the user, and mobility aids of the user.

According to an aspect of another embodiment, there is provided an apparatus for estimating disability information based on user image data analysis, the apparatus including a characteristic extraction unit configured to obtain first image data of a user, and extract user characteristic information including at least one of appearance characteristics, behavior characteristics, or object characteristics associated with the user from the first image data, and an analysis unit configured to infer disability information including at least one of a disability type or a disability degree of the user on the basis of the user characteristic information.

In an embodiment, the apparatus for estimating disability information based on user image data analysis may include a control unit configured to set at least one of a first element associated with a user input application method of an information terminal to be manipulated by the user or a second element associated with an information output method of the information terminal on the basis of the disability information.

In an embodiment, the characteristic extraction unit may determine whether the user accesses the information terminal, and derives the user characteristic information according to the determination result.

In an embodiment, the apparatus for estimating disability information based on user image data analysis may include a manipulation evaluation unit configured to obtain second image data obtained by capturing an image of the user who manipulates the information terminal, extract the behavior characteristics from the second image data, and evaluate a user's manipulation ability for the information terminal on the basis of the extracted behavior characteristics.

According to an aspect of another embodiment, there is provided a customized information providing system based on disability information of a user, the system including a disability information estimation apparatus configured to obtain first image data of a user, extract user characteristic information including at least one of appearance characteristics, behavior characteristics, or object characteristics associated with the user from the first image data, and infer disability information including at least one of a disability type or a disability degree of the user on the basis of the user characteristic information, and an information terminal in which a setting of at least one of a first element associated with a user input application method and a second element associated with an information output method on the basis of the disability information is adjusted so that the user interacts therewith.

In an embodiment, the disability type comprises at least one of a visual impairment type, a hearing-impaired type, or a physical disability type.

In an embodiment, the appearance characteristics comprise at least one of height information or posture information of the user, and the behavior characteristics comprise information on at least one of a user's gait, gaze, or movement of a preset body part.

In an embodiment, the object characteristics comprise information on clothes worn by the user, items carried by the user, and mobility aids of the user.

The above-described means for solving problems are merely illustrative, and should not be construed as limiting the disclosure. In an embodiment to the exemplary embodiments described above, additional embodiments may exist in the drawings and detailed description of disclosure.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic configuration view of a customized information providing system based on disability information of a user according to an embodiment of the disclosure;

FIG. 2 is a conceptual view for describing a process of estimating the disability information using image data obtained by capturing an image of a user;

FIG. 3 is a conceptual view for describing a process of estimating the disability information the user in view of a shape attribute in the image data including a cane-type object carried by the user;

FIG. 4 is a conceptual view for describing a process of selectively allowing a user to access a braille cell module;

FIG. 5 is a schematic configuration view of an apparatus for estimating disability information based on user image data analysis according to an embodiment of the disclosure;

FIG. 6 is an operation flowchart of a disability information estimation method based on user image data analysis according to an embodiment of the disclosure; and

FIG. 7 is an operation flowchart of a process of evaluating a user's manipulation ability for an information terminal by analyzing behavior characteristics when the user manipulates the information terminal, according to an embodiment of the disclosure.

### DETAILED DESCRIPTION

Hereinafter, embodiments of the disclosure will be described in detail with references to the accompanying drawings in such a manner that those of ordinary skill in the art to which the disclosure pertains can easily implement them. However, the disclosure may be implemented in several different forms and is not limited to the embodiments described herein. In an embodiment, in order to clearly explain the disclosure in the drawings, portions irrelevant to the descriptions are omitted, and similar reference numerals are attached to similar portions throughout the specification.

Throughout this specification, when a part is “connected” with another part, it is not only “directly connected” but also “electrically connected”, or “indirectly connected” with another element interposed therebetween.

Throughout this specification, when it is said that a member is located “on”, “on an upper portion of”, “on a top of”, “under”, “on a lower portion of”, and “on a bottom of” another member, this means that a member is located on the other member. It includes not only the case where they are in contact, but also the case where another member exists between two members.

Throughout this specification, when a portion or a part “includes” a component or an element, it means that other components or elements may be further included, rather than excluding other components, unless otherwise stated.

The disclosure relates to an apparatus and method for evaluating disability information based on user image data analysis.

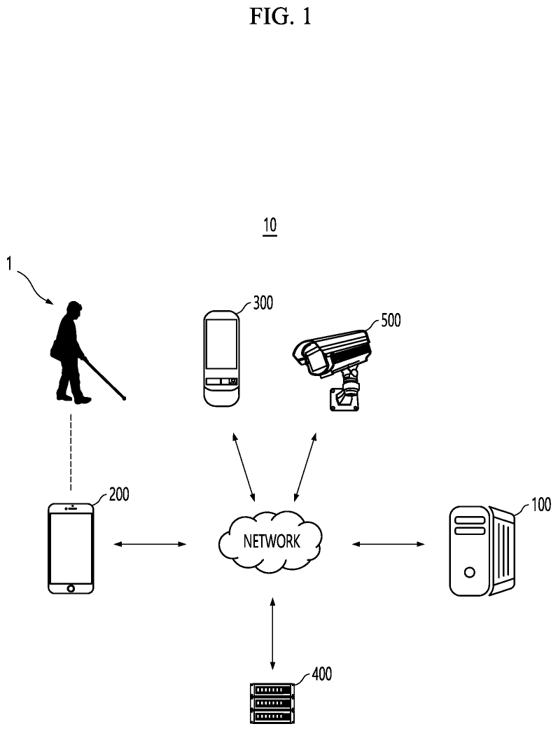

FIG. 1 is a schematic configuration view of a customized information providing system based on disability information of a user according to an embodiment of the disclosure.

Referring to FIG. 1, a customized information providing system 10 based on disability information of a user (hereinafter, referred to as “information providing system 10”) according to an embodiment of the disclosure may include a disability information estimation apparatus 100 based on user image data analysis (hereinafter referred to as “disability information estimation apparatus 100”) according to an embodiment of the disclosure, a user terminal 200, an information terminal 300, an external server 400, and an image capturing device 500.

The disability information estimation apparatus 100, the user terminal 200, the information terminal 300, the external server 400, and the image capturing device 500 may communicate with each other via a network 20. The network 20 refers to a connection structure which enables information to be exchanged between each node, such as terminals and servers. Examples of the network 20 may include a 3^{rd} Generation Partnership Project (3GPP) network, Long Term Evolution (LTE) network, 5G network, World Interoperability for Microwave Access (WIMAX) network, Internet, Local Area Network (LAN), Wireless Local Area Network (Wireless LAN), Wide Area Network (WAN), Personal Area Network (PAN), a wifi network, a Bluetooth network, a satellite broadcasting network, an analog broadcasting network, a Digital Multimedia Broadcasting (DMB) network, or the like. However, the network 20 is not limited to those examples described above.

In an embodiment, although not shown in the figures, the information providing system 10 may include an auxiliary terminal (not shown) carried by a disabled user or the like. The auxiliary terminal (not shown) may be, e.g., a device capable of outputting braille-based information for a visually impaired user (e.g., a blind person or the like). In an example embodiment, the device capable of outputting braille-based information may include a braille watch, a braille pad, or the like. which has a display including a plurality of braille protrusion members. However, the auxiliary terminal is not limited to the device for the visually impaired user. Thus, the auxiliary terminal (not shown) herein may further include a wide range of various devices that can be used to assist various information vulnerable users such as disabled users and elderly users. For example, the auxiliary terminal (not shown) may be a concept including various devices for assisting the user's vision, mobility, hearing, cognitive ability, and the like, such as a hearing aid, an interpreter, a cane, an electric wheelchair, and the like. In an embodiment, according to the implementation of the disclosure, a user interacting with the information terminal 300 may have (possess) only the user terminal 200 without a separate auxiliary terminal (not shown) or have (possess) both the auxiliary terminal (not shown) and the user terminal 200.

The user terminal 200 may include any type of wireless communication device, e.g., a smartphone, a smart pad, a tablet PC, and the like, and terminals for PCS (Personal Communication System), GSM (Global System for Mobile communication), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), and Wibro (Wireless Broadband Internet).

For reference, in relation to the embodiment of the disclosure, a ‘target space’ mainly means a multi-use facility or space used by a large number of people, such as subway stations, public transportation platforms, train stations, airports, shopping malls, department stores, parks, movie theaters, schools, stadiums, gyms, public institutions, and the like. However, the target space is not limited to those described above.

In an embodiment, the information terminal 300 may be installed in the target space for the purpose of providing the user with route information for destinations, such as major facilities (toilets, elevators, escalators, or the like), stores (shops), and specific positions, or the like in the target space. However, the information terminal 200 is not limited to those described above, and may also be installed for the purpose of providing the user (passerby) with information on the target space or various types of information that is not limited to the target space.

In an embodiment, in the description of the embodiment of the disclosure, the information terminal 300 may be a concept that broadly includes an unmanned device, a kiosk, a digital signage, and the like. In an embodiment, as will be described in detail below, the information terminal 300 disclosed herein may be designed to provide a user interface (UI)/user experience (UX) in a customized manner according to user information, in consideration of information accessibility of the users corresponding to the information vulnerable users, such as the disabled user, the elderly user, and the like.

In an embodiment, in the description of the embodiment of the disclosure, the disability information estimation apparatus 100 may be configured to mount in the information terminal 300, and may be a subcomponent of the information terminal 300 for performing an operation of controlling (changing) elements associated with the UI/UX of the information terminal 300. In another example, according to the embodiment of the disclosure, the disability information estimation apparatus 100 may be a device that is provided separately from the information terminal 300 and configured to perform an operation of controlling (changing) the elements associated with the UI/UX of the information terminal 300.

In the description of the embodiment of the disclosure, the external server 400 may be configured to retain user information of each user in advance and provide necessary user information to the disability information estimation apparatus 100. In an embodiment, the external server 400 may have a function of providing spatial information (e.g., positions of major facilities, map information, or the like) on the target space, in which the information terminal 300 with which the user interacts is located, to the disability information estimation apparatus 100. In an example embodiment, the external server 400 may retain the user information in advance in a manner of receiving user information of a user who has (possesses) the corresponding user terminal 200 from the user terminal 200 in advance and storing the user information as user information for the corresponding user.

In an embodiment, according to an embodiment of the disclosure, the user information that is secured in advance by the external server 400 and provided to the disability information estimation apparatus 100 may include personal information such as a user's name, age, gender, address, and contact information, as well as disability information such as a disability type and a disability grade (level) in the case of a disabled user.

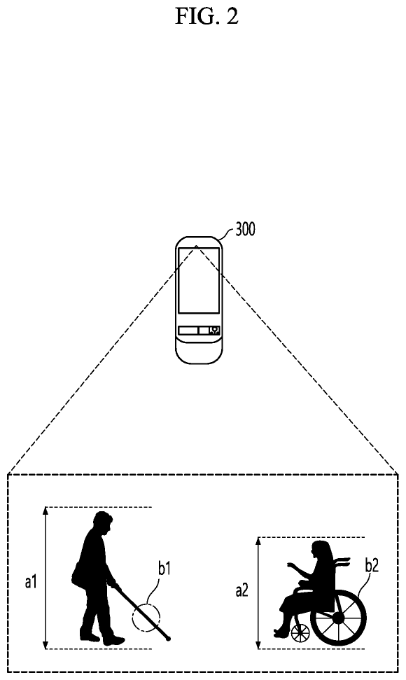

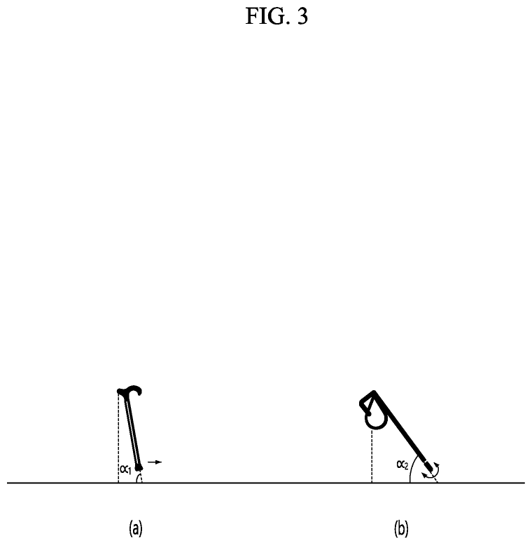

Hereinafter, an embodiment of the disclosure, in which the disability information estimation apparatus 100 estimates disability information including the presence or absence (the existence) of disability, a disability type, a disability degree, and the like of the corresponding user through analysis of image data obtained by capturing an image of the user, will be described in detail with reference to FIGS. 2 and 3.

The disability information estimation apparatus 100 may obtain disability information including a disability type and a disability degree of the user. In this regard, in the description of the embodiment of the disclosure, the disability type of the user may include at least one of a visual impairment type, a hearing-impaired type, and a physical disability type. In an embodiment, in the description of the embodiment of the disclosure, the disability degree of the user may include information indicating a degree of deterioration in physical ability corresponding to the disability type of the user, and may refer to, for example, disability degree information according to Criteria for Judgment by Disability Type, but the disability degree of the user is not limited to the disability degree information. In an embodiment, according to an embodiment of the disclosure, when a particular user is a user having a combination of different types of disabilities, the disability information on the corresponding user may be obtained in the form of complex information including a plurality of disability types and a disability degree according to each of the disability types.

The disability information estimation apparatus 100 may estimate the disability information by analyzing image data obtained by capturing an image of the corresponding user. In an example embodiment, the disability information estimation apparatus 100 may obtain data on an image that is captured by an infrastructure (not shown) such as a closed circuit television (CCTV) provided in a space in which the user interacts (more particularly, an act of operating the target device by the user and an act of providing information that the user wants to obtain through the target device) with the target device, or may obtain data on an image, which is captured in a process in which a specific user accesses the information terminal 300 by a camera module (not shown) provided in the information terminal 300 or captured in a process in which the specific user manipulates the information terminal 300 by a peripheral part of the information terminal 300, from the information terminal 300.

FIG. 2 is a conceptual view for describing a process of estimating the disability information using image data obtained by capturing an image of a user.

Referring to FIG. 2, the disability information estimation apparatus 100 may operate to estimate the disability information from at least one of appearance characteristics, behavior characteristics, and object characteristics of the user reflected in first image data. For reference, in the description of the embodiment of the disclosure, “first image data” and “second image data” may be terms classified according to whether the image data is captured while the user manipulates the information terminal 300.

More particularly, the first image data may refer to image data captured at a time point before the user reaches the information terminal 300 or captured at a position irrelevant to the information terminal 300, and the second image data may refer to image data captured in a state in which the user manipulates (interacts with) the information terminal 300 after reaching the information terminal 300.

According to an embodiment of the disclosure, the appearance characteristics of the user may include the height, appearance, physique, and the like of the user. In an embodiment, the behavior characteristics of the user may include the gait, a body movement, and the like of the user. In an embodiment, the object characteristics of the user may include identification of a tool, the user terminal 200, an auxiliary terminal (not shown), clothes, a walking assistance aids, a wheelchair, and the like which the user possess.

In an example embodiment, referring to FIG. 2, the disability information estimation apparatus 100 may obtain height information a1 of a first user (the user shown on a left side of FIG. 2) reflected in the image data, object information b1 indicating that the user is holding a cane, height information a2 of a second user (the user shown on a right side of FIG. 2) reflected in the image data, and object information b2 indicating that the user is riding in a wheelchair, and may use the pieces of height information a1 and a2 and the pieces of object information b1 and b2 to estimate the existence of disability, a disability type, a disability degree, and the like of each user.

In another example, in the case of a visually impaired user who wears “sunglasses” and walks while carrying a “white cane”, the disability information estimation apparatus 100 may identify the sunglasses, the white cane, and the like as the object characteristics of the corresponding user from the image in which the corresponding user appears, and may recognize that the corresponding user is a visually impaired user in response to the identification. Similarly, the disability information estimation apparatus 100 may recognize that the user is a visually impaired user by identifying characteristics in which the user walks while striking guidance blocks on the ground with the white cane as the behavior characteristics. In this regard, the disability information estimation apparatus 100 may have a pre-trained artificial intelligence model (e.g., a deep learning algorithm that detects an appearance object through semantic segmentation or the like) for detecting the appearance characteristics, the behavior characteristics, the object characteristics, and the like from the received image (first image data).

In other words, the disability information estimation apparatus 100 may extract user characteristic information including at least one of the appearance characteristics, which include at least one piece of information on the user's height and posture, the behavior characteristics, which include pieces of information on the user's gait, gaze, and movement of a preset body part, and object characteristics, which include pieces of information on clothes worn by the user, items carried by the user, and mobility aids of the user, from the first image data.

In an embodiment, the disability information estimation apparatus 100 may operate to estimate the user's disability information reflected in the first image data by comparing the type of the user characteristic information, which is identified from the first image data, with characteristic pattern data, which is pre-configured to represent characteristic patterns for each of the disability type and the disability degree.

For example, the disability information estimation apparatus 100 may have in advance a table including items, such as wearing sunglasses, holding a cane, walking after checking obstacles in a proceeding direction with a cane, moving along braille leading blocks, and the like as the characteristic pattern data for the characteristic patterns of blind (visually impaired) users, and when all of the above-described items are detected from the first image data, or when it is detected that the number of items is greater than or equal to a preset threshold, the disability information estimation apparatus 100 may estimate that the corresponding user is a blind (visually impaired) user. Of course, the detection items for each of the disability type and the disability degree are not limited to the above-described examples and may be modified in various forms to broadly reflect the characteristics of the disabled user.

FIG. 3 is a conceptual view for describing a process of estimating the disability information the user in view of a shape attribute in the image data including a cane-type object carried by the user.

Referring to FIG. 3, when it is determined that the user reflected in the first image data carries a cane by the object characteristics, the disability information estimation apparatus 100 according to an embodiment of the disclosure may operate to estimate that the corresponding user is a visually impaired user, who moves while holding a white cane, in view of the specific behavior characteristics of the user using the cane, or estimate that the corresponding user is a physically disabled (mobility impaired) user who has difficulty in walking and moves while supporting the body with a cane.

Particularly, referring to FIG. 3, the disability information estimation apparatus 100 may estimate the disability information in such a manner that as an angle between the cane-type object and the ground is relatively large (α₁>α₂), the probability that the corresponding user corresponds to the physically disabled user increases.

In an embodiment, referring to FIG. 3, the disability information estimation apparatus 100 may estimate the disability information in consideration of a correlation between a moving direction of the cane-type object and a proceeding direction of the user. For example, when the proceeding direction of the user and the moving direction of the cane-type object are relatively close to each other in the first image data as shown in FIG. 3A, the disability information may be estimated such that the probability that the corresponding user corresponds to a physically disabled user increases. In an embodiment, when the similarity between the proceeding direction of the user and the moving direction of the cane-type object is relatively small as the user moves the cane while swinging the cane left and right sides in front of the user's proceeding direction as shown in FIG. 3B, the disability information may be estimated such that the probability that the corresponding user corresponds to a visually impaired user, who walks while checking obstacles in front of the moving direction of the white cane by using the white cane.

When the disability information of the user is obtained through the analysis of the first image data as described above, the disability information estimation apparatus 100 may set at least one of a first element associated with a user input application method of the target device, such as the information terminal 300, the user terminal 200, or the like, and a second element associated with an information output method of the target device, on the basis of the obtained disability information.

In the description of the embodiment of the disclosure, the first element associated with the user input application method of the target device may include at least one of a touch-based input element, a voice-based input element, and an auxiliary means input element, and the second element associated with the information output method for the user may include at least one of a visual output element, an auditory output element, and a braille-based output element.

In other words, the disability information estimation apparatus 100 may adjust the first element and the second element, which are elements associated with the UI/UX of the target device, in a customized manner by considering the user's reduced physical ability on the basis of the disability information including the disability type and disability degree of the user, so that the corresponding user can interact with the target device.

More particularly, the setting of the first element and the second element of the information terminal 300 in a customized manner may include providing a customized UI/UX for the user by changing the conventional information input/output method, in which the usability of the disabled user or the like is not taken into consideration, for the information terminal 300 by considering the information access ability of the user, such as the user's vision, mobility, hearing, cognitive ability, and the like on the basis of the disability type of the user, so that pieces of predefined information can be provided (transmitted) through the target device such as the information terminal 300 in a manner that the user can conveniently manipulate and easily understand.

That is, the term “customized setting” in the disclosure may include a conversion rule for converting at least some of the elements related to the conventional UI/UX of the information terminal 300, so that the information terminal 300 can provide information (contents), which is the same as that previously provided for non-disabled users and the like, in a form in which the information vulnerable users can conveniently manipulate and easily understand.

When the user for the disability information is a visually impaired user, the disability information estimation apparatus 100 may change the first element of the target device so that the target device is controlled on the basis of at least one control input application method from among a braille input method, a voice input method, and an intuitive touch input method.

In an embodiment, when the user for the disability information is a visually impaired user, the disability information estimation apparatus 100 may change the second element of the target device into a customized setting in which predetermined information is displayed on the basis of at least one information output method from among a voice output method and a braille output method.

More particularly, for a customized setting including the braille input method as the control input application method and the braille output method as the information output method, the disability information estimation apparatus 100 may control the information terminal 300 so that the user can access a braille cell module 310 provided in the information terminal 300.

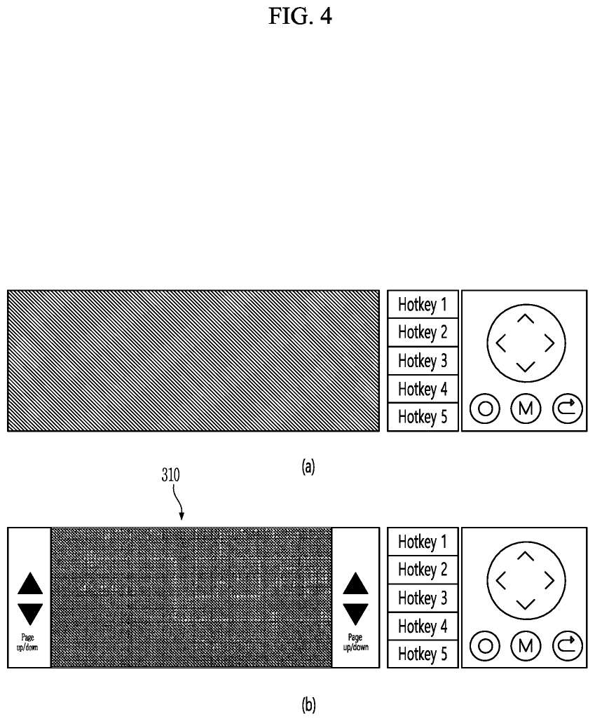

FIG. 4 is a conceptual view for describing a process of selectively allowing a user to access a braille cell module.

Referring to FIG. 4, the braille cell module 310 may be placed in a locked state (the state shown in FIG. 4A) by a predefined locker (e.g., a cover member that restricts an access to the braille cell module 310 in a closed state, or the like) so that a plurality of braille protrusions provided in the braille cell module 310 are prevented from being contaminated and damaged by preventing the braille cell module 310 from being used by users other than the visually impaired user, and the locked state may be selectively released (the state shown in FIG. 4B) only when the visually impaired user intends to use the information terminal 300. Accordingly, when it is determined that the corresponding user corresponds to the visually impaired user on the basis of the disability information of the corresponding user, the disability information estimation apparatus 100 may release the locker of the braille cell module 310.

For example, when it is determined that the visually impaired user is located away from the information terminal 300 by a distance within a preset distance range on the basis of position information of the user, or it is determined that an expected arrival time of the user to the information terminal 300 calculated on the basis of the position information of the user is within a preset time range, the disability information estimation apparatus 100 may control the information terminal 300 to release the above-described locker.

In other words, when it is determined that the user is a blind user on the basis of the disability information of the user, the disability information estimation apparatus 100 may control the target device to allow the corresponding user to access the braille cell module 310.

In an embodiment, in relation to the intuitive touch input method, the disability information estimation apparatus 100 may receive a control input related to entering (selecting) and moving to a menu through a touch display provided in the information terminal 300 on the basis of a menu tree that is preset for the information provided by the information terminal 300, and here, the disability information estimation apparatus 100 may receive the control input of the user by utilizing the entire region of the touch display instead of receiving the control input on the basis of buttons, icons, and the like that are locally displayed on the touch display.

In an example embodiment, the disability information estimation apparatus 100 may apply a customized setting in such a configuration that the user can enter (select), move to, or the like the menu provided in the information terminal 300 by a simplified touch control method, which can be performed even by the visually impaired user with sufficient ease and convenience and includes a control input for touching at least a portion of an inner region of the touch display once or more, a control input for dragging (sliding) at least a portion of the inner region of the touch display, and the like.

In an embodiment, in this regard, the disability information estimation apparatus 100 may set up various manipulation methods for the information terminal 300 in a customized manner on the basis of the menu tree preset according to the information provided by the information terminal 300. For example, the manipulation methods may include a manipulation of the information terminal 300 performed in accordance with the number of touches onto the touch display, a manipulation of the information terminal performed in match with a dragging (sliding) direction (e.g., an up-down direction, a horizontal direction, a diagonal direction between the upper left and lower right corners, a diagonal direction between the upper right and lower left corners, or the like) on the touch display, and the like.

In an embodiment, the disability information estimation apparatus 100 may guide the user with specific details of the intuitive touch input method set before the user starts to interact with the information terminal 300. In an example embodiment, the disability information estimation apparatus 100 may control the information terminal 300 to provide descriptions for the intuitive touch input, which is set correspondently to the visually impaired user in the information terminal 300, through voice guidance.

In an embodiment, in relation to the voice input method, the disability information estimation apparatus 100 may apply a customized setting for converting a content input method for a text-type entry item into the voice input method to the information terminal 300, so that text information (e.g., contents of items to be entered by typing on the screen provided to non-disabled users, or the like) requested for input while the visually impaired user manipulates the information terminal 300 is received by the disability information estimation apparatus 100 through a voice input by the visually impaired user.

In an example embodiment, when it is determined that the user has entered an item (or menu) into which text information is requested to input, the disability information estimation apparatus 100 may activate a voice recognition module (e.g., a microphone or the like) of the information terminal 300, and may output a signal requesting utterance through voice. When the user's voice is applied, the input to the corresponding entry item may be processed by extracting text information corresponding to the corresponding voice through the voice recognition module.

In an embodiment, the disability information estimation apparatus 100 may apply, to the information terminal 300, a customized setting including a function to enable manipulations such as entering (selecting), moving to, or the like the menu provided by the information terminal 300 through the voice control input.

In an embodiment, in relation to the voice output method, the disability information estimation apparatus 100 may apply a customized setting to the information terminal 300 in such a configuration in which menu information related to the information provided by the information terminal 300, visual information displayed on a screen in a predetermined situation, and the like are converted into voice and the corresponding voice is provided. For example, in response to the user's entry into the menu, the disability information estimation apparatus 100 may provide a list of menus that the user can select or a list of menu-related control manipulations to the user by arranging the list by the voice output method on the basis of the menu tree. In an embodiment, the disability information estimation apparatus 100 may apply a customized setting to the information terminal 300 in a such configuration that text information or the like, which is displayed for a user other than the visually impaired user, included in the screen of the information terminal 300 is converted into voice by the voice output method and provided to the user.

In an embodiment, in relation to the braille output method, the disability information estimation apparatus 100 may convert (braille translation) the menu information, the text information, or the like, which is related to the information provided from the information terminal 300, into braille text, and provide the braille text through the braille cell module 310.

In an embodiment, when the user for the disability information is a hearing-impaired user, the disability information estimation apparatus 100 may change the first element and the second element of the matched information terminal 300 to a customized setting in which the matched information terminal 300 is controlled on the basis of at least one control input application method from among a touch input method and a gesture input method.

In an embodiment, when the user for the disability information is a hearing-impaired user, the disability information estimation apparatus 100 may change elements associated with the UI/UX of the matched information terminal 300 to a customized setting in which the information terminal 300 displays information on the basis of at least one information output method from among an intuitive visual information output method and a gesture output method.

More particularly, in relation to the gesture input method, the disability information estimation apparatus 100 may apply, to the information terminal 300, a customized setting including a function of controlling the information terminal 300 on the basis of a gesture input by activating the camera module (not shown) of the information terminal 300 and extracting the contents of the user's intention from the image secured through the camera module (not shown), so that the intention of the hearing-impaired user based on a gesture-based communication means of the hearing-impaired user, such as sign language, spoken language (oral method), or the like are identified.

In an embodiment, in relation to the intuitive visual information output method, the disability information estimation apparatus 100 may apply a customized setting to the information terminal 300 in such a configuration that menu information, text information, and the like conventionally provided by the information terminal 300 in connection with information provided through the information terminal 300 are replaced with intuitive visual information, such as intuitive symbols, icons, and pictograms corresponding to the conventional menu information, text information, and the like, or displayed together with the intuitive visual information, so that even illiterate users or the like understand the information provided by the information terminal 300.

In this regard, the disability information estimation apparatus 100 may include a first conversion algorithm for deriving the intuitive visual information that matches the menu information, the text information, and the like provided by the information terminal 300. For example, the first conversion algorithm may search the external server 400 in which words, vocabularies, and expressions matching the intuitive visual information, such as the symbols, the icons, the pictograms, and the like are combined with each other and stored together, and may select the intuitive visual information corresponding to the menu information, the text information, and the like provided by the information terminal 300. Then, the searched intuitive visual information may be reflected in the customized setting.

In an embodiment, in relation to the gesture output method, the disability information estimation apparatus 100 may support the hearing-impaired user to understand the information provided from the information terminal 300 in such a way that a sign language (sign language), a spoken language (oral method), or the like, which matches the menu information, the text information, and the like associated with the information terminal 300, is output.

In an example embodiment, the disability information estimation apparatus 100 may include a second conversion algorithm for converting the menu information, the text information, and the like associated with the information terminal 300 into the gesture output, such as a sign language, so that predetermined information is provided by the gesture output method. More particularly, the second conversion algorithm may include a tool for generating gesture-based avatar images corresponding to the menu information, the text information, and the like associated with the information terminal 300. The disability information estimation apparatus 100 may apply a customized setting to the information terminal 300 in such a configuration that the gesture-based avatar image generated from the text information, which corresponds to the menu information chosen by the user or corresponds to information requested by the user, by the second conversion algorithm is displayed on the information terminal 300.

In an embodiment, in this regard, when the disability type of the user is a hearing-impaired type, the disability information estimation apparatus 100 may control a method to displaying sign language image information, which includes information corresponding to a user input applied to the target device, on the basis of user status information including at least one of position information, body information, and gaze information associated with the user.

More particularly, the disability information estimation apparatus 100 may adjust the specification (enlargement/reduction degree) and position, in which the sign language image information or oral image information to be described below is displayed, of a display of the target device, on the basis of the user status information. For example, the disability information estimation apparatus 100 may finely adjust the specification and position of the output image information on the basis of the body information, the gaze information, and the like of the user, so that the hand part of the avatar included in the sign language image information or the face part of the user included in the oral image information is located at a position corresponding to a user's eye level in the display region of the target device.

In an embodiment, according to an embodiment of the disclosure, the disability information estimation apparatus 100 may control a timing, at which the sign language image information or the oral image information is displayed on the display or enlarged and displayed on the display, according to the size of the output information provided to the user through the target device.

In an embodiment, according to an embodiment of the disclosure, the disability information estimation apparatus 100 may operate so that the second element is determined to adopt an information transmission method familiar to the hearing-impaired user from among speech reading (oral method), in which the hearing-impaired user understands what the other person is saying by looking at the movement of the speaker's lips and facial expression, and sign language (sign language method), which is a language for transmitting the meaning by changing the shape of the hand and fingers, the direction of the palm, the position of the hand, and the movement of the hand, and the avatar generated in advance selectively outputs the sign language image information, which is expressed through hand gestures, or the oral image information, in which the face region of the pre-generated avatar is enlarged so that the user can easily recognize the change in the shape of the mouth and understand the output information, on the basis of the determined second element.

In this regard, when it is determined that the user is of the hearing-impaired type, the disability information estimation apparatus 100 may determine the second element to be based on the sign language method or the oral method on the basis of a user input applied to the target device. Alternatively, the disability information estimation apparatus 100 may estimate the information transmission method with which the hearing-impaired user is familiar from among the sign language method and the oral method by evaluating whether the user can understand the avatar-based output image, which is output by the sign language method or the oral method according to an initial setting, through the analysis of the user's image data, or analyzing gaze information on each of local regions constituting the output image, and may set the second element in a customized manner to match the estimation result.

For example, when it is determined that the user is of the hearing-impaired type, the disability information estimation apparatus 100 may determine the second element as the avatar image of the sign language method according to the initial (basic) setting and output predetermined information, and when it is determined that the ratio of the gaze of the user, who views the avatar image, to the lips region of the avatar is equal to or greater than a preset threshold ratio, the disability information estimation apparatus 100 may operate to re-output the same information with the avatar image of the oral method by estimating that the corresponding user is a user relatively familiar with oral method communication.

In an embodiment, according to an embodiment of the disclosure, the disability information estimation apparatus 100 may apply a customized setting to the information terminal 300 in such a configuration that the visibility of various information provided from the information terminal 300 may be improved in consideration of dim-sighted users (e.g., the elderly users) having at least some residual vision. In an example embodiment, the disability information estimation apparatus 100 may apply a customized setting to the information terminal 300 in such a configuration that various types of visual information displayed on the display, such as texts, images, and media, are provided by magnifying them or improving the contrast, sharpness, resolution, brightness, and the like thereof compared to the conventional ones.

In an embodiment, when the user for the disability information is a physically disabled user, the disability information estimation apparatus 100 may apply a customized setting to the target device in such a configuration that the information terminal 300 is controlled on the basis of at least one control input application method from among the touch input method using a lower interface of the display, a physical key input method, and the gesture input method.

In still another example, when the disability type of the user is a physical disability type, the disability information estimation apparatus 100 may control the position and driving method of the auxiliary means input element (e.g., various device manipulation means such as physical keys and a track ball) provided in the target device on the basis of the user status information including at least one of the position information, the body information, and the gaze information associated with the user.

More particularly, the interface, which is displayed on the touch display of the information terminal 300 in a normal state, may include a main region, a touch manipulation region, and an advertising region that are arranged in the up-down direction, so that the normal user conveniently manipulates the touch manipulation region in a standing state. Meanwhile, for the physically disabled user such as a wheelchair occupant, a customized setting may be applied to the information terminal 300 in such a configuration that the lowermost advertisement region is controlled to be removed or moved upward, and a graphic UI is changed so that the touch manipulation region is placed at the lower portion of the display.

In this regard, the position of the touch manipulation region, which is a region of the touch display of the information terminal 300 for receiving the touch input, may be appropriately determined by the customized setting set for the physically disabled user such as the wheelchair occupant, and In an embodiment, the positions of the display and the auxiliary input device may be appropriately changed by through a separate lifter, so that various usability for the physically disabled user may be given to the information terminal 300 disclosed in the disclosure in such a way that the physically disabled user can use the touch display or the auxiliary input device (e.g., physical keys). In an embodiment, the position change control of the touch manipulation region and the control of the lifter (not shown) are applied not only to the physically disabled user such as the wheelchair occupant, but also to child users with small heights, so that the information terminal 300, of course, can improve the use conveniences.

In summary, the disability information estimation apparatus 100 may obtain the user input, which is applied to the target device by the user on the basis of the first element set on the basis of the disability information, and may output information corresponding to the user input through the target device on the basis of the second element.

In an embodiment, the disability information estimation apparatus 100 obtain second image data obtained by capturing an image of the user who manipulates the information terminal 300. Particularly, the disability information estimation apparatus 100 may receive the second image data, which is captured by a camera module provided in the information terminal 300, from the information terminal 300.

In an embodiment, the disability information estimation apparatus 100 may extract behavior characteristics of the user, who manipulates the information terminal 300, from the obtained second image data, and evaluate a user's manipulation ability for the information terminal 300 on the basis of the extracted behavior characteristics.

For example, when it is determined that the user does not perform a subsequent manipulation on the information terminal 300 for more than a preset first threshold time in a state in which a screen, to which a user input for entering a predetermined menu or selecting a predetermined item is applied, is displayed on the display of the information terminal 300, the disability information estimation apparatus 100 may determine that the corresponding user does not accurately understand the information currently displayed on the display of the information terminal 300 and thus may evaluate the user's manipulation ability for the information terminal 300 to be low.

In another example, the disability information estimation apparatus 100 may evaluate the user's manipulation ability by determining whether the corresponding user understands the information displayed on the display of the current information terminal 300, in view of the behavior characteristics, which include a proximity between the user's face and an information display region (a display screen or the like.) of the information terminal 300, a change in the position where the user is gazed, an utterance through voice, and the like, reflected in the second image data.

In a more specific example, when it is determined that the corresponding user continues to gaze at a position on the display, which is irrelevant to an information output region including the menu display position, the position selected through the user input, or the like, for more than a preset second threshold time after the screen, to which the user input for entering a predetermined menu or selecting a predetermined item is applied, is displayed on the display of the information terminal 300, the disability information estimation apparatus 100 may determine that the corresponding user does not accurately understand the information currently displayed on the display of the information terminal 300 and thus may evaluate the manipulation ability of the corresponding user to be low.

In still another example, when it is determined that an area or a ratio of the region, which is occupied by the user's face region, in the second image data is increased as the user moves his or her face to be relatively close to the display screen of the information terminal 300 after the screen to which, the user input for entering a predetermined menu or selecting a predetermined item is applied, is displayed on the display of the information terminal 300, the disability information estimation apparatus 100 may determine that it is difficult for the corresponding user to visually check the information currently displayed on the display (in other words, the manipulation ability is evaluated to be low) and thus may control the information terminal 300 in a customized manner so that the size (the size, specification, or the like of a font) of the currently displayed information is increased.

In an embodiment, according to an embodiment of the disclosure, the disability information estimation apparatus 100 may operate to check whether the user's previously identified disability information, which is received from the user terminal 200 of the user or received from the external server 400, matches the disability information derived based on a result of analyzing the image data obtained by capturing an image of the corresponding user. For example, when it is necessary to verify that a particular user is actually a disabled user, the disability information estimation apparatus 100 may perform a function of verifying whether the disability information of the corresponding user is stolen information by comparing the user's disability information provided (presented) through a separate route such as the user terminal 200, the external server 400, and the like, and disability information estimated based on a result of image data-based analysis for the actual appearance of the corresponding user and actions performed by the corresponding user.

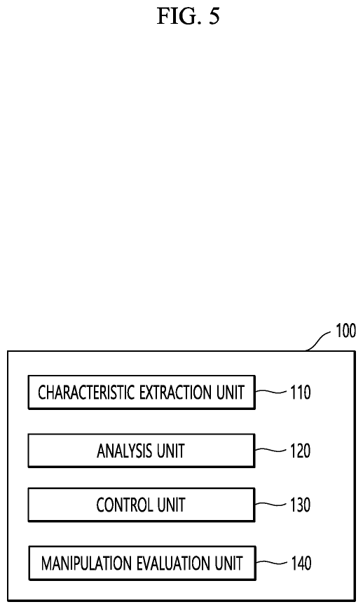

FIG. 5 is a schematic configuration view of an apparatus for estimating disability information based on user image data analysis according to an embodiment of the disclosure.

Referring to FIG. 5, the disability information estimation apparatus 100 may include a characteristic extraction unit 110, an analysis unit 120, a control unit 130, and a manipulation evaluation unit 140.

The characteristic extraction unit 110 may obtain first image data of a user. In an example embodiment, the characteristic extraction unit 110 may receive, as the first image data, at least one of image data captured through a camera module provided in the information terminal 300 and image data obtained by an infrastructure (not shown) configured to capture a target space in which the user is located.

The characteristic extraction unit 110 may extract user characteristic information including at least one of appearance characteristics, behavior characteristics, and object characteristics associated with the user from the first image data.

According to an embodiment of the disclosure, the characteristic extraction unit 110 may operate to preferentially determine whether the user accesses the information terminal 300, and selectively derive the user characteristic information according to the determination result.

The analysis unit 120 may infer disability information including a disability type and a disability degree of the corresponding user on the basis of the user characteristic information derived by the characteristic extraction unit 110.

The control unit 130 may set at least one of a first element associated with a user input application method of the information terminal 300 to be manipulated by the corresponding user and a second element associated with an information output method of the information terminal 300 on the basis of the user's disability information estimated by the analysis unit 120.

The manipulation evaluation unit 140 may obtain second image data obtained by capturing an image of the user who manipulates the information terminal 300. In an embodiment, the manipulation evaluation unit 140 may extract behavior characteristics of the user from the obtained second image data.

In an embodiment, the manipulation evaluation unit 140 may evaluate a user's manipulation ability for the information terminal 300 on the basis of the behavior characteristics derived from the second image data.

Hereinafter, the operation flow of the disclosure will be briefly described based on the details given above.

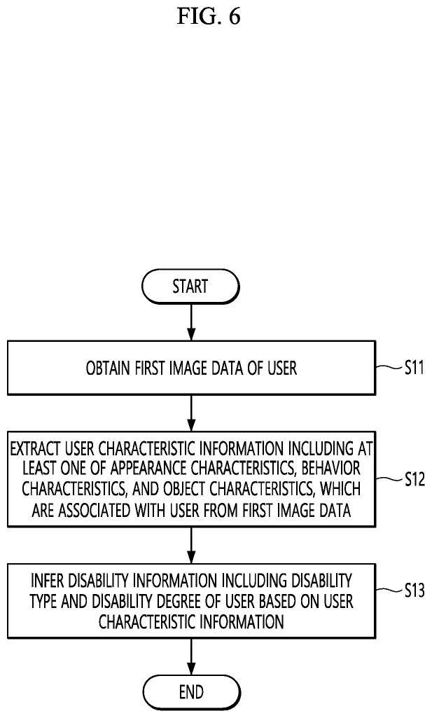

FIG. 6 is an operation flowchart of a disability information estimation method based on user image data analysis according to an embodiment of the disclosure.

The disability information estimation method based on the user image data analysis illustrated in FIG. 6 may be performed by the above-described disability information estimation apparatus 100. Thus, although omitted below, the contents described with respect to the disability information estimation apparatus 100 may be equally applied to a description of the disability information estimation method based on user image data analysis.

Referring to FIG. 6, in operation S11, the characteristic extraction unit 110 may obtain first image data of a user.

In detail, according to an embodiment of the disclosure, in operation S11, the characteristic extraction unit 110 may receive, as the first image data, at least one of image data captured through a camera module provided in the information terminal 300 and image data obtained by an infrastructure (not shown) configured to capture an image of a target space in which the user is located.

Next, in operation S12, the characteristic extraction unit 110 may extract user characteristic information including at least one of appearance characteristics, behavior characteristics, and object characteristics associated with the user from the first image data obtained in operation S11.

According to an embodiment of the disclosure, in operation S12, the characteristic extraction unit 110 may preferentially determine whether the user accesses the information terminal 300, and selectively derive the user characteristic information according to the determination result.

Next, in operation S13, the analysis unit 120 may infer disability information including a disability type and a disability degree of the corresponding user on the basis of the user characteristic information derived in operation S12.

In the descriptions given above, operations S11 to S13 may be further divided into additional operations or be combined into fewer operations, according to an embodiment of the disclosure. In an embodiment, some operations may be omitted if necessary, and the order between the operations may be changed.

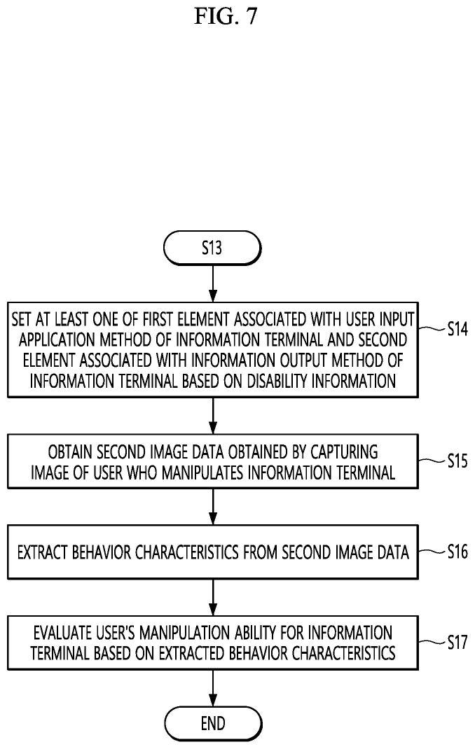

FIG. 7 is an operation flowchart of a process of evaluating a user's manipulation ability for an information terminal by analyzing behavior characteristics when the user manipulates the information terminal, according to an embodiment of the disclosure.

The process, which is shown in FIG. 7, of evaluating the manipulation ability for the information terminal by analyzing behavior characteristics when the user manipulates the information terminal may be performed by the above-described disability information estimation apparatus 100. Thus, although omitted below, the contents described with respect to the disability information estimation apparatus 100 may be equally applied to a description of FIG. 7.

Referring to FIG. 7, in operation S14, the control unit 130 may set at least one of a first element associated with a user input application method of the information terminal 300 to be manipulated by the corresponding user and a second element associated with an information output method of the information terminal 300 on the basis of the user's disability information estimated in operation S13.

Next, in operation S15, the manipulation evaluation unit 140 may obtain second image data obtained by capturing an image of the user who manipulates the information terminal 300.

Next, in operation S16, the manipulation evaluation unit 140 may extract behavior characteristics of the user from the second image data.

Next, in operation S17, the manipulation evaluation unit 140 may evaluate a user's manipulation ability for the information terminal 300 on the basis of the behavior characteristics derived in operation S16.

In the descriptions given above, operations S14 to S17 may be further divided into additional operations or be combined into fewer operations, according to an embodiment of the disclosure. In an embodiment, some operations may be omitted if necessary, and the order between the operations may be changed.

The disability information estimation method based on user image data analysis according to an embodiment of the disclosure may be recorded in a computer-readable medium in the form of program instructions that can be executed by various computers. The computer-readable medium described above may include program instructions, data files, data structures, and the like which may be used alone or in combinations thereof. The program instructions recorded in the medium above described may include program instructions that are specially designed and configured only for the disclosure and program instructions that are known and available to those skilled in the art of computer software. The computer-readable recording medium may include hardware devices specially configured to store and execute program instructions. Examples of the hardware devices may include magnetic media such as a hard disk, a floppy disk and a magnetic tape (Magnetic Media), optical media such as a CD-ROM and a DVD (Optical Media), and magneto-optical media (Magneto-optical Media) such as a floppy disk (Floptical Disk), a ROM, a RAM, and flash memory. Examples of the program instructions may include high-level language codes executed by a computer using an interpreter or the like as well as machine language codes such as those generated by a compiler. The hardware devices described above may be configured to operate as one or more software modules so as to perform the operations of the disclosure, and vice versa.

In an embodiment, the above-described disability information estimation method based on user image data analysis may be implemented in the form of a computer program or application that is stored on a recording medium and executed by a computer.

According to the above-described problem solving means of the disclosure, it is possible to provide an apparatus and method for evaluating disability information based on user image data analysis, capable of inferring the existence of disability, a disability type, a disability degree, or the like of a user in view of characteristics such as an appearance, a behavior, an object, or the like reflected in image data of the corresponding user.

According to the above-described problem solving means of the disclosure, it is possible to provide a customized information providing system capable of detecting a situation in which a user has difficulty in manipulation of an information terminal such as a kiosk, an unmanned device, or the like, on the basis of an analysis result of image data for a user interacting with the corresponding information terminal, and assisting the user in response thereto.

However, the effects obtainable from the disclosure are not limited to the above-described effects, and other effects may exist.

The foregoing descriptions of the disclosure described above are illustrative of example embodiments, and those of ordinary skill in the art to which the disclosure pertains will understand that it can be easily modified into other specific forms without changing the technical spirit or essential features of the disclosure. Therefore, it should be understood that the embodiments described above are illustrative in all respects and not restrictive. For example, each component described as a single type may be implemented in a distributed manner, and likewise components described as distributed may also be implemented in a combined form.

The scope of the disclosure is indicated by the following claims rather than the above detailed description, and all changes or modifications derived from the meaning and scope of the claims and their equivalent concepts should be construed as being included in the scope of the disclosure.

What is claimed is:

1. A method of estimating disability information based on user image data analysis, the method comprising:
obtaining first image data of a user;
extracting, from the first image data, user characteristic information comprising at least one of appearance characteristics, behavior characteristics, or object characteristics, which are associated with the user; and
inferring disability information comprising at least one of a disability type or a disability degree of the user on the basis of the user characteristic information.

2. The method of claim 1, wherein the disability type comprises at least one of a visual impairment type, a hearing-impaired type, or a physical disability type.

3. The method of claim 2, further comprising setting at least one of a first element associated with a user input application method of an information terminal to be manipulated by the user or a second element associated with an information output method of the information terminal on the basis of the disability information.

4. The method of claim 3, wherein
in the extracting of the user characteristic information,
whether the user accesses the information terminal is determined, and
the user characteristic information is derived according to a determination result.

5. The method of claim 3, further comprising:
obtaining second image data obtained by capturing an image of the user who manipulates the information terminal;
extracting the behavior characteristics from the second image data; and
evaluating a user's manipulation ability for the information terminal on the basis of the extracted behavior characteristics.

6. The method of claim 3, wherein
in the obtaining of the first image data,
at least one of image data captured through a camera module provided in the information terminal or image data obtained by an infrastructure configured to capture an image of a target space, in which the user is located, is received as the first image data.

7. The method of claim 1, wherein
the appearance characteristics comprise at least one of height information or posture information of the user, and
the behavior characteristics comprise information on at least one of a user's gait, gaze, or movement of a preset body part.

8. The method of claim 1, wherein the object characteristics comprise information on clothes worn by the user, items carried by the user, and mobility aids of the user.

9. An apparatus for estimating disability information based on user image data analysis, the apparatus comprising:
a characteristic extraction unit configured to obtain first image data of a user, and extract user characteristic information comprising at least one of appearance characteristics, behavior characteristics, or object characteristics associated with the user from the first image data; and
an analysis unit configured to infer disability information comprising at least one of a disability type or a disability degree of the user on the basis of the user characteristic information.

10. The apparatus of claim 9, wherein the disability type comprises at least one of a visual impairment type, a hearing-impaired type, or a physical disability type.

11. The apparatus of claim 10, further comprising a control unit configured to set at least one of a first element associated with a user input application method of an information terminal to be manipulated by the user or a second element associated with an information output method of the information terminal on the basis of the disability information.

12. The apparatus of claim 11, wherein the characteristic extraction unit determines whether the user accesses the information terminal, and derives the user characteristic information according to the determination result.

13. The apparatus of claim 11, further comprising a manipulation evaluation unit configured to obtain second image data obtained by capturing an image of the user who manipulates the information terminal, extract the behavior characteristics from the second image data, and evaluate a user's manipulation ability for the information terminal on the basis of the extracted behavior characteristics.

14. The apparatus of claim 9, wherein
the appearance characteristics comprise at least one of height information or posture information of the user, and
the behavior characteristics comprise information on at least one of a user's gait, gaze, or movement of a preset body part.

15. The apparatus of claim 9, wherein
the object characteristics comprise information on clothes worn by the user, items carried by the user, and mobility aids of the user.

16. A customized information providing system based on disability information of a user, the customized information providing system comprising:
a disability information estimation apparatus configured to obtain first image data of a user, extract user characteristic information comprising at least one of appearance characteristics, behavior characteristics, or object characteristics associated with the user from the first image data, and infer disability information comprising at least one of a disability type or a disability degree of the user on the basis of the user characteristic information; and
an information terminal of which a setting of at least one of a first element associated with a user input application method and a second element associated with an information output method on the basis of the disability information is adjusted to enable interaction with the user.

17. The system of claim 16, wherein the disability type comprises at least one of a visual impairment type, a hearing-impaired type, or a physical disability type.

18. The system of claim 16, wherein
the appearance characteristics comprise at least one of height information or posture information of the user, and
the behavior characteristics comprise information on at least one of a user's gait, gaze, or movement of a preset body part.

19. The system of claim 16, wherein the object characteristics comprise information on clothes worn by the user, items carried by the user, and mobility aids of the user.
